(19) United States
(12) Patent Application Publication
(10) Pub. No.: US 2026/0034338 A1
(43) Pub. Date: Feb. 5, 2026

(54) BALLOONS FOR BALLOON CATHETERS

(71) Applicant: EDWARDS LIFESCIENCES CORPORATION, Irvine, CA (US)

(72) Inventors: David Maimon, Atlit (IL); Tamir S. Levi, Zikhron Yaakov (IL)

(21) Appl. No.: 19/355,145

(22) Filed: Oct. 10, 2025

Related U.S. Application Data
(63) Continuation of application No. PCT/US2024/025366, filed on Apr. 19, 2024.
(60) Provisional application No. 63/497,206, filed on Apr. 20, 2023.

(51) Int. Cl. A61M 25/10; A61F 2/24

(52) U.S. Cl. CPC A61M 25/10; A61F 2/2433; A61M 2025/1081

(57) ABSTRACT

A balloon catheter can comprise a shaft and a balloon coupled to a distal end portion of the shaft. The balloon can comprise a main body having first and second end portions and a sleeve. The sleeve can comprise a first end portion extending partially over the first end portion of the balloon and a second end portion extending partially over an adjacent surface of the balloon catheter. The first end portion of the sleeve can comprise a first plurality of circumferentially spaced, axially extending fingers. The second end portion of the sleeve can comprise an intermediate portion and a second plurality of fingers extending in a direction opposite the first plurality of fingers.

### CROSS REFERENCE TO RELATED APPLICATION

This application is a continuation of PCT Patent Application No. PCT/US2024/025366 filed on Apr. 19, 2024, which application claims the benefit of U.S. Provisional Patent Application No. 63/497,206, filed Apr. 20, 2023, each of these applications being incorporated by reference herein in its entirety.

### FIELD

The present disclosure relates to balloon catheters for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples, the balloon of the delivery apparatus can tear during an implantation procedure, such as from inadvertent overinflation. A tear in a lateral or circumferential direction across the balloon can result in a portion of the balloon separating from the balloon catheter and/or the balloon becoming caught on the medical implant being implanted with the delivery apparatus, or on another component the delivery apparatus when removing the delivery apparatus from a body of a patient.

Accordingly, a need exists for improved balloons for balloon catheters and methods of fabricating such balloons that can avoid lateral tears if the balloon should fail during a procedure.

### SUMMARY

Described herein are balloon catheters for use in medical procedures. The disclosed balloon catheters can, for example, be configured to tear in an axial direction under pressure from an inflation pressure received by a catheter balloon, thereby simplifying retrieval and removal of torn catheter balloons from the patient's body. As such, the devices disclosed herein can, among other things, overcome one or more of the deficiencies of known balloon catheters. In some examples, balloon catheters disclosed herein can comprise delivery apparatuses that are adapted to delivery and deploy a medical implant (for example, a stent or prosthetic valve) inside a patient's body.

A balloon catheter can comprise a handle and one or more shafts coupled to the handle.

In some examples, the balloon catheter can comprise a catheter balloon mounted on a distal end portion of one of the shafts.

In some examples, the catheter balloon can comprise a first end portion and a second end portion.

In some examples, the balloon catheter can comprise a sleeve.

In some examples, the sleeve can comprise a first end portion extending partially over the first end portion of the balloon and a second end portion extending partially over an adjacent surface of the balloon catheter.

In some examples, the first end portion of the sleeve can comprise a first plurality of circumferentially spaced, axially extending fingers.

In some examples, the second end portion can comprise a cylindrical portion.

In some examples, the cylindrical portion can have a first radial thickness, each finger of the first plurality of fingers can have a second radial thickness, and the first radial thickness can be less than the second radial thickness.

In some examples, the cylindrical portion can comprise an axially extending perforation.

In some examples, the second end portion can comprise an intermediate portion and a second plurality of circumferentially spaced, axially extending fingers extending in a direction opposite the first plurality of fingers.

In some examples, each of the first plurality of fingers can be aligned with a corresponding one of the second plurality of fingers in a circumferential direction.

In some examples, the catheter balloon can comprise a plurality of circumferentially spaced ribs extending axially along the first end portion of the main body.

In some examples, each of the plurality of ribs can have a first end disposed toward the first end portion of the main body and a second end disposed toward the second end portion of the main body.

In some examples, the plurality of ribs can converge at their first ends.

In some examples, each of the plurality of ribs can taper from the first end to the second end of the rib.

In some examples, each of the plurality of ribs can taper in circumferential width.

In some examples, each of the plurality of ribs can taper in radial thickness.

In some examples, the main body can further comprise an inner surface and an outer surface disposed radially outwards of the inner surface.

In some examples, the plurality of ribs can extend from the inner surface of the main body in a radially inwards direction.

In some examples, the plurality of ribs can extend from the outer surface of the main body in a radially outwards direction.

In some examples, the plurality of ribs can be disposed between the inner surface and the outer surface of the main body.

In some examples, a balloon catheter can comprise a shaft having a proximal end portion and a distal end portion and an inflatable balloon mounted on the distal end portion of the shaft. The balloon can comprise a main body having first and second end portions and a sleeve. The sleeve can comprise a first end portion extending partially over the first end portion of the balloon and a second end portion extending partially over an adjacent surface of the balloon catheter. The first end portion of the sleeve can comprise a plurality of circumferentially spaced, axially extending fingers.

In some examples, a sleeve for a catheter balloon can comprise a cylindrical portion configured to be disposed around an adjacent surface of the balloon catheter and a plurality of circumferentially spaced, axially extending fingers extending from the cylindrical portion.

In some examples, a balloon catheter can comprise a shaft having a proximal end portion and a distal end portion and an inflatable balloon mounted on the distal end portion of the shaft. The inflatable balloon can comprise a main body and a plurality of circumferentially spaced ribs extending axially along at least a portion of the main body. Each of the plurality of ribs can have a first width at a first end thereof and a second width at a second end thereof, wherein the first width can be greater than the second width.

In some examples, a balloon catheter can comprise a shaft having a proximal end portion and a distal end portion and an inflatable balloon mounted on the distal end portion of the shaft. The inflatable balloon can comprise a main body having first and second end portions and a plurality of circumferentially spaced ribs extending axially along the first end portion of the main body. Each rib can have a first end disposed toward the first end portion of the main body and a second end disposed toward the second end portion of the main body. The ribs can converge at their first ends.

In some examples, a delivery apparatus comprises one or more of the components recited in Examples 1-30 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The above method(s) can be performed on a living animal or on a simulation, such as on a cadaver, cadaver heart, anthropomorphic ghost, simulator (for example, with body parts, heart, tissue, etc. being simulated).

### BRIEF DESCRIPTION OF THE DRAWINGS

FIG. 1 is a perspective view of a prosthetic heart valve, according to one example.

FIG. 2 is a perspective view of a delivery apparatus for a prosthetic heart valve, according to one example.

FIG. 3A is a side view of a distal end portion of the delivery apparatus of FIG. 2 comprising a catheter balloon and a catheter balloon sleeve, according to one example.

FIG. 3B is an enlarged side view of the catheter balloon sleeve of FIG. 3A.

FIG. 4A is a side view of the distal end portion of a delivery apparatus similar to the delivery apparatus of FIG. 3A showing an axial tear starting to form along a distal end portion of the catheter balloon.

FIG. 4B is a side view of the distal end portion of the delivery apparatus similar to the delivery apparatus of FIG. 3A showing the axial tear propagated along a length of the distal end portion of the catheter balloon.

FIG. 5A is a side view of a distal end portion of a delivery apparatus comprising a catheter balloon and first and second catheter balloon sleeves, according to one example.

FIG. 5B is an enlarged side view of the first catheter balloon sleeve of FIG. 5A.

FIG. 5C is an enlarged side view of the second catheter balloon sleeve of FIG. 5A.

FIG. 6A is a side view of a distal end portion of a delivery apparatus comprising a catheter balloon and a catheter balloon sleeve, according to one example.

FIG. 6B is an enlarged side view of the catheter balloon sleeve of FIG. 6A.

FIG. 7 shows a side view of a distal end portion of a delivery apparatus comprising a catheter balloon and two catheter balloon sleeves, according to one example.

FIG. 8 shows a side view of a distal end portion of the delivery apparatus comprising a catheter balloon, according to one example.

FIG. 9 shows a side view of a distal end portion of a delivery apparatus comprising a catheter balloon, according to one example.

FIG. 10 shows a side view of a distal end portion of a delivery apparatus comprising a catheter balloon, according to one example.

FIG. 11 shows a cross-section of a distal end portion of the delivery apparatus of FIG. 8, according to a first example.

FIG. 12 shows a cross-section of a distal end portion of the delivery apparatus of FIG. 8, according to a second example.

FIG. 13 shows a cross-section of a distal end portion of the delivery apparatus of FIG. 8, according to a third example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, the term “axial direction” refers to a direction that is parallel to a central longitudinal axis of an object, such as a balloon catheter or a catheter balloon.

As used herein, the terms “radial direction” and “lateral direction” refer to a direction that extends radially outward from the central longitudinal axis of the object (such as a catheter balloon) and is perpendicular to the axial direction.

As used herein, the term “circumferential direction” refers to a direction along a circumference of the object (such as a catheter balloon).

As used herein, the term “radial thickness” refers to a radial thickness of the object (such as a main body of the catheter balloon) measured from an inner circumferential surface to an outer circumferential surface of the object.

As used herein, “e.g.” means “for example,” and “i.e.” means “that is.”

### Overview of the Disclosed Technology

Described herein are examples of balloon catheters that can be used in various medical procedures. In some examples, the disclosed balloon catheters can comprise a delivery apparatus that can be used to navigate a subject's vasculature to deliver an implantable, expandable medical device (for example, a prosthetic heart valve), tools, agents, or other therapy to a location within the body of a subject. Examples of procedures in which the catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like. In some examples, the disclosed balloon catheters can be used for performing procedures for opening or widening a blood vessel or heart valve annulus, such as an angioplasty or a valvuloplasty.

During a medical procedure involving a balloon catheter, the balloon of the catheter is inflated (such as to deploy a prosthetic valve or another type of implant) by injecting an inflation fluid under pressure into the balloon, and then deflated by withdrawing the inflation fluid from the balloon. Thereafter, the catheter is retracted through an introducer sheath and removed from the patient's body. A catheter balloon sometimes can rupture or tear during the medical procedure, such as if the balloon is inadvertently overinflated, which can complicate retrieval and removal of the catheter from the patient's body. For example, if a balloon forms a tear in the circumferential direction, there may be a greater risk of torn portions of the balloon becoming detached from the balloon catheter.

To address one or more problems of known catheter balloons, it would be desirable to control how a catheter balloon ruptures when subjected to overinflation. For example, it would be desirable to design a catheter balloon such that if it is subjected to overinflation and rupture occurs, the balloon will form one or more axially extending tears.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

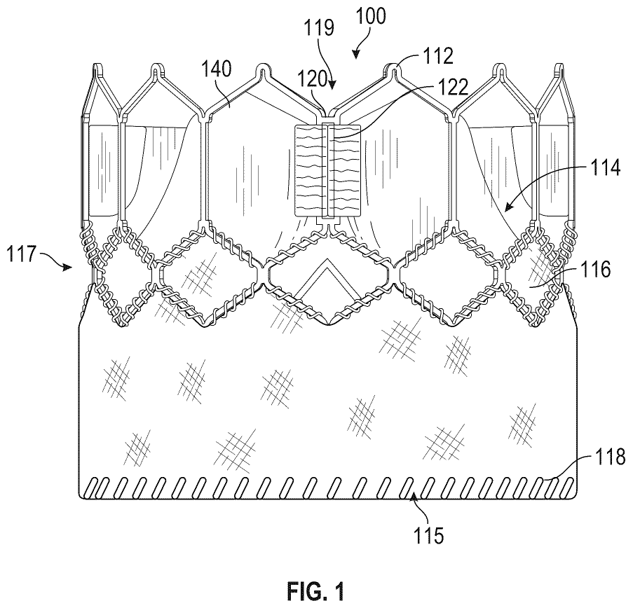

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic valve 100 can comprise a frame 112, a valvular structure 114, an inner skirt 116, and a perivalvular outer sealing member or outer skirt 118. The prosthetic valve 100 can comprise an inflow end portion 115 and an outflow end portion 119, and an intermediate portion 117 extending therebetween.

The valvular structure 114 can comprise a plurality of leaflets 140 collectively forming a leaflet structure. In some examples, the valvular structure 114 can comprise three leaflets 140 arranged in a tricuspid arrangement. However, there can be a greater or fewer number of leaflets 140. The leaflets can be secured to one another at their adjacent sides to form commissures 122 of the valvular structure 114. The lower edge of the valvular structure 114 can have an undulating, curved scalloped shape, and can be secured to the inner skirt 116 by sutures (not shown). In some examples, the leaflets 140 can be formed of pericardial tissue (such as bovine pericardial tissue), biocompatible synthetic materials, or other various suitable natural or synthetic materials as known in the art and described in U.S. Pat. No. 6,730,118, which is incorporated by reference herein.

The frame 112 can be made of any of various suitable plastically-expandable materials (for example, stainless steel, etc.) or self-expanding materials (for example, Nitinol) as known in the art. When constructed of a plastically-expandable material, the frame 112 (and thus the valve 100) can be crimped to a radially compressed state on a delivery catheter and then expanded inside a patient by an inflatable balloon or equivalent expansion mechanism. When constructed of a self-expandable material, the frame 112 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 112) include, metal alloys, polymers, or combinations thereof. Example metal alloys can comprise one or more of the following: nickel, cobalt, chromium, molybdenum, titanium, or other biocompatible metal. In some examples, the frame 112 can comprise stainless steel. In some examples, the frame 112 can comprise cobalt-chromium. In some examples, the frame 112 can comprise nickel-cobalt-chromium. In some examples, the frame 112 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The inner skirt 116 and/or the outer skirt 118 can be wholly or partly formed of any suitable biological material, synthetic material (for example, any of various polymers), or combinations thereof. In some examples, the inner skirt 116 and/or the outer skirt 118 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the inner skirt 116 and/or the outer skirt 118 can comprise a fabric without interlaced yarns or fibers or randomly interlaced yarns or fibers (sometimes referred to as non-woven fabrics), such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the inner skirt 116 and/or the outer skirt 118 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the inner skirt 116 and/or the outer skirt 118 can comprise a sponge material or foam, such as polyurethane foam. In some examples, the inner skirt 116 and/or the outer skirt 118 can comprise natural tissue, such as pericardium (for example, bovine pericardium, porcine pericardium, equine pericardium, or pericardium from other sources).

### Delivery Apparatus

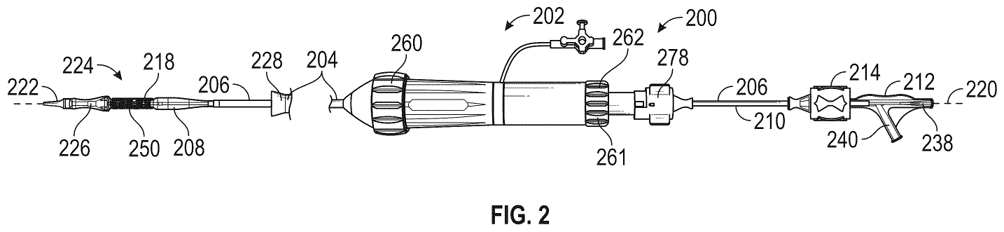

FIG. 2 shows a delivery apparatus 200, according to one example, in the form of a balloon catheter that can be used to implant a prosthetic medical device. In some examples, the delivery apparatus 200 can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic heart valve into a heart.

The delivery apparatus 200 in the illustrated example of FIG. 2 comprises a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic heart valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable catheter balloon 218 (which also referred to herein as a “balloon”) of the delivery apparatus 200. A distal end portion of the inner shaft 208 can extend distally beyond the distal end portion of the intermediate shaft 206.

The catheter balloon 218 can be coupled to the distal end portion of the intermediate shaft 206.

In some examples, a distal end of the catheter balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the catheter balloon 218 can overlay a valve mounting portion 224 of a distal end portion of the delivery apparatus 200 and a distal end portion of the catheter balloon 218 can overly a distal shoulder 226 of the delivery apparatus 200. The valve mounting portion 224 and the intermediate portion of the catheter balloon 218 can be configured to receive a prosthetic heart valve in a radially compressed state. For example, as shown schematically in FIG. 2, a prosthetic heart valve 250 (which can be one of the prosthetic heart valves described herein) can be mounted around the catheter balloon 218, at the valve mounting portion 224 of the delivery apparatus 200.

The balloon shoulder assembly, including the distal shoulder 226, is configured to maintain the prosthetic heart valve 250 (or other prosthetic medical device) at a fixed position on the catheter balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic heart valve 250 is mounted in the radially compressed state on the valve mounting portion 224 (as shown in FIG. 2) and during delivery of the prosthetic heart valve to the target implantation site. As such, the distal tip portion 228 can be configured to resist movement of the prosthetic heart valve 250 relative to the catheter balloon 218 proximally, in the axial direction, relative to the catheter balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the catheter balloon 218. As such, fluid from the fluid source can flow to the fluid passageway from the annular space to inflate the catheter balloon 218 and radially expand and deploy the prosthetic heart valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Publication No. WO2022/046585, which is incorporated by reference herein.

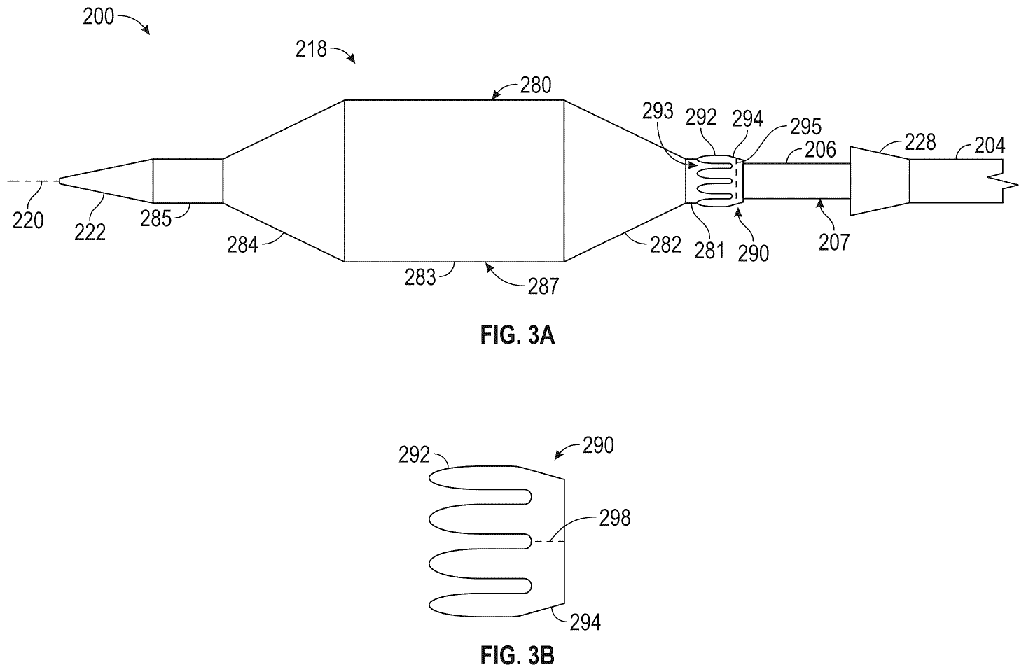

FIG. 3A is a side view of a distal end portion of the delivery apparatus 200 comprising the catheter balloon 218 and a catheter balloon sleeve 290 (which may also be referred to herein as a “sleeve”), according to one example. The catheter balloon 218 can comprise an inflatable main body 280 configured to be inflatable between a deflated state and an inflated state. The main body 280 can comprise a proximal neck portion 281, a proximal conical portion 282 distally disposed relative to the proximal neck portion 281, an intermediate portion 283 distally disposed relative to the proximal conical portion 282, a distal conical portion 284 distally disposed relative to the intermediate portion 283, and a distal neck portion 285 distally disposed relative to the distal conical portion 284. In some examples, the proximal neck portion 281 and the distal neck portion 285 can be the respective proximal and distal end portions of the catheter balloon 218.

The proximal neck portion 281 can be coupled to a shaft (such as the intermediate shaft 206) of the delivery apparatus 200, such as with an adhesive or by welding. The proximal conical portion 282 can taper from the intermediate portion 283 to the proximal neck portion 281. The intermediate portion 283 can be configured to receive a prosthetic heart valve (such as the prosthetic heart valve 100) in a radially compressed state. The intermediate portion 283 can be a cylindrical portion of the catheter balloon 218. The distal conical portion 284 can taper from the intermediate portion 283 to the distal neck portion 285. The distal neck portion 285 can be coupled to the nose cone 222 or another distal end component of the delivery apparatus 200 (such as a distal shoulder), such as with an adhesive or by welding. In some examples, the shaft 206 can extend all the way through the catheter balloon 218 to the nose cone 222, in which case the distal neck portion 285 and proximal neck portion 281 can both be secured to the intermediate shaft 206. In some examples, the proximal neck portion 281, the proximal conical portion 282, the intermediate portion 283, the distal conical portion 284, and the distal neck portion 285 can be integrally formed as a single component.

The main body 280 can further comprise an outer surface 287 that defines an exterior of the catheter balloon 218.

The main body 280 of the catheter balloon 218 can be formed of any of various suitable thermoplastics and thermoset polymers. Examples of thermoplastics include polyolefins, polyamides, such as nylon 12, nylon 11, nylon 6/12, nylon 6, and nylon 66, polyesters, polyethers, polyurethanes, polyureas, polyvinyls, polyacrylics, fluoropolyirers, copolymers and block copolyrmers thereof, such as block copolyrmers of polyether and polyamide, for example, Pebax®; and mixtures thereof. Examples of thermosets include elastomers such as EPDM, epichlorohydrin, nitrile butadiene elastomers, silicones, etc. Thermosets, such as epoxies and isocyanates, can also be used. Biocompatible thermosets may also be used, and these include, for example, biodegradable polycaprolactone, poly(dimethylsiloxane) containing polyurethanes and ureas, and polysiloxanes. Any of the balloons disclosed herein can be made of one or more of any of these types of materials.

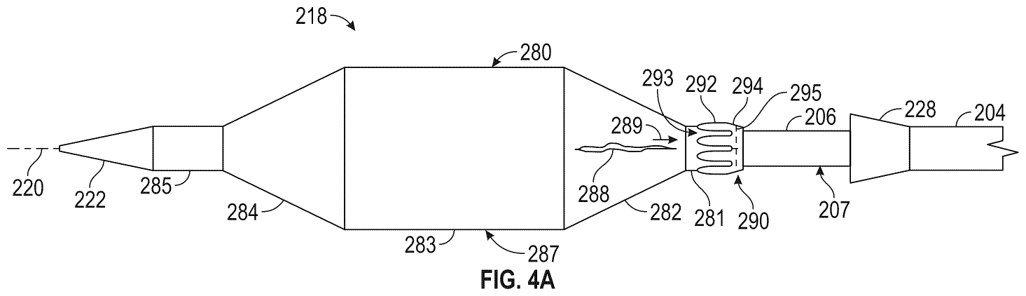

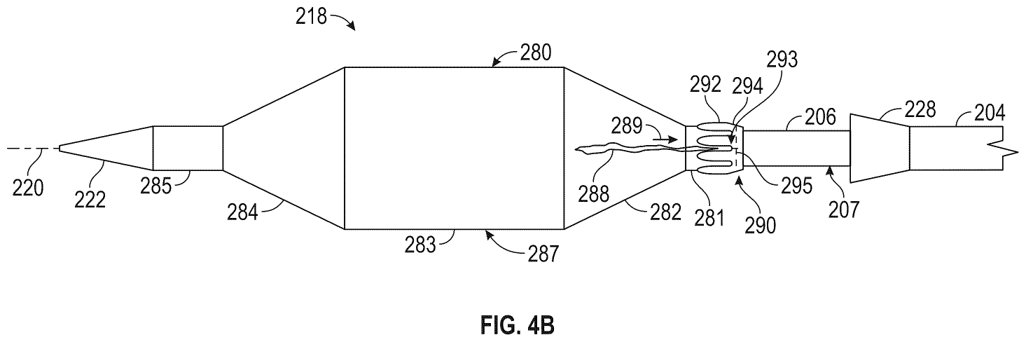

In the illustrated example, the delivery apparatus 200 further comprises the sleeve 290. The sleeve 290 can comprise a first end portion comprising a plurality of axially extending fingers 292 and a second end portion comprising a cylindrical portion 294. When coupled to the delivery apparatus 200, the sleeve 290 can help control the formation of tears in the catheter balloon 218. For example, when the sleeve 290 is coupled to the proximal neck portion 281 of the catheter balloon 218, a tear (FIGS. 4A-4B) formed at the proximal neck portion 281 can be more likely to propagate in the longitudinal or axial direction than in the lateral or circumferential direction.

The plurality of fingers 292 can comprise any number of circumferentially spaced fingers, such as two fingers, three fingers, four fingers, five fingers, six fingers, seven fingers, eight fingers, nine fingers, ten fingers, etc. Each of the plurality of fingers 292 can extend in the axial direction. For example, each of the plurality of fingers can extend in the distal direction from the cylindrical portion 294.

Each of the plurality of fingers 292 can have an axial length. In the illustrated example, each of the plurality of fingers 292 has the same axial length. In some examples, a first set of the plurality of fingers 292 can have a first axial length and a second set of the plurality of fingers 292 can have a second axial length different than the first axial length.

Each of the plurality of fingers 292 can have a radial thickness. In some examples, each of the plurality of fingers 292 can have the same radial thickness. In some examples, a first set of the plurality of fingers 292 can have a first radial thickness and a second set of the plurality of fingers 292 can have a second, different radial thickness. In some examples, at least one of the plurality of fingers 292 can have a uniform radial thickness along its axial length. In some examples, at least one of the plurality of fingers 292 can taper in the axial direction (for example, in the distal direction).

Each of the plurality of fingers 292 can have a circumferential width. In the illustrated example, each of the plurality of fingers 292 has the same circumferential width. In some examples, a first set of the plurality of fingers 292 can have a first circumferential width and a second set of the plurality of fingers 292 can have a second, different circumferential width. In some examples, at least one of the plurality of fingers 292 can have a uniform circumferential width along its axial length. In some examples, at least one of the plurality of fingers 292 can taper in the axial direction (for example, in the distal direction).

Since the plurality of fingers 292 are circumferentially spaced apart, adjacent ones of the plurality of fingers 292 can form a gap 293 (which is also referred to herein as a “slit” or a “slot”) therebetween. The gap 293 can extend in the axial direction. In some examples, each gap 293 between circumferentially adjacent ones of the plurality of fingers 292 can have the same circumferential width. In some examples, a first set of gaps 293 can have a first circumferential width and a second set of gaps 293 can have a second circumferential width different than the first circumferential width.

The plurality of fingers 292 can be configured to extend partially over an end portion of the catheter balloon 218. In the illustrated example, the plurality of fingers 292 can be arranged in a circumferential direction around the proximal neck portion 281 such that each of the plurality of fingers 292 extends along an axial length of the proximal neck portion 281. In some examples, the plurality of fingers 292 can extend along the entire axial length of the proximal neck portion 281. In some examples, the plurality of fingers 292 can be additionally or alternatively configured to extend over a portion of the distal end portion of the intermediate shaft 206. In some examples, the plurality of fingers 292 can be additionally or alternatively configured to extend over a portion of the proximal conical portion 282 and/or the intermediate portion 283 of the catheter balloon 218.

The cylindrical portion 294 can comprise an annular structure. The cylindrical portion 294 can extend partially over an adjacent surface of the delivery apparatus 200. In the illustrated example, the cylindrical portion 294 extends partially over the proximal neck portion 281 and partially over the outer surface 207 of the intermediate shaft 206 such that a proximal terminal end 295 of the neck portion 281 is located underneath and covered by the cylindrical portion 294. The sleeve 290 (including the fingers 292 and the cylindrical portion 294) can be affixed to the outer surface 287 of the catheter balloon 218 and the outer surface 207 of the intermediate shaft 206 using any suitable techniques and mechanisms, such as an adhesive, welding, or mechanical fasteners. In some examples, the cylindrical portion 294 can extend over the entirety of the proximal neck portion 281. In some examples, the cylindrical portion 294 does not extend over the intermediate shaft 206 such that an entirety of the cylindrical portion 294 is located on the balloon (for example, on the neck portion 281). In some examples, the plurality of fingers 292 can additionally or alternatively extend at least partially over the proximal conical portion 282 and/or the intermediate portion 283.

Each of the plurality of fingers 292 can extend from the cylindrical portion 294 in the axial direction. In the illustrated example, each of the plurality of fingers 292 extends in the distal direction from the cylindrical portion 294 and are coupled to the cylindrical portion 294. The plurality of fingers 292 can be coupled to the cylindrical portion 294 using adhesives, mechanical fasteners, stitches, ultrasonic welding, etc. In some examples, the plurality of fingers 292 and the cylindrical portion 294 can be integrally formed as a unitary structure, such as by molding. In some examples, the catheter balloon 218, the plurality of fingers 292, and the cylindrical portion 294 can be integrally formed as a unitary structure.

In some examples, each of the plurality of fingers 292 can have a first radial thickness and the cylindrical portion 294 can have a second radial thickness. In some examples, the first radial thickness can be greater than the second radial thickness. In such examples, the cylindrical portion 294 can be configured to tear before the plurality of fingers 292, thereby better enabling axial tears to propagate across the cylindrical portion 294 in the axial direction instead of across the fingers 292 in the circumferential direction.

The plurality of fingers 292 can be adhered to the outer surface 287 of the catheter balloon 218. In the illustrated example, the plurality of fingers 292 can be adhered to the outer surface 287 of the catheter balloon 218 at the proximal neck portion 281. In some examples, the plurality of fingers 292 can additionally or alternatively be adhered to the outer surface 287 of the catheter balloon 218 along the proximal conical portion 282 and/or the intermediate portion 283. However, the plurality of fingers 292 can be attached using any suitable attachment method (for example, mechanical fasteners, ultrasonic welding, etc.) at any suitable location on the outer surface 287 of the catheter balloon 218.

In some examples, the cylindrical portion 294 can optionally comprise one or more weakened regions that are more likely to tear compared to an adjacent portion of the cylindrical region 294. As shown in FIG. 3B, the weakened region can be a section of the cylindrical portion that includes one or more perforations 298. In lieu of or in addition to the perforations 298, the one or more weakened regions can comprise a region that is radially thinner than adjacent portions of the cylindrical region 294, such as may be formed by an axially extending score line formed in those regions. The perforations 298 can be arrayed in the axial direction along an axial length or the entire axial length of the cylindrical portion 294. The perforations 298 can be circumferentially aligned between circumferentially adjacent ones of the plurality of fingers 292. Since the region that includes the perforation 298 can be structurally weaker than adjacent portions of the cylindrical portion 294, the cylindrical portion 294 can be configured to first tear along the perforations 298. This can beneficially allow an axial tear to propagate across the cylindrical portion 294 in the axial direction. In some examples, the cylindrical portion 294 can be configured to tear at the perforations 298 when the catheter balloon 218 is subject to a predetermined pressure or amount of force due to overinflation. In some examples, the cylindrical portion 294 can comprise a plurality of sets of perforations 298, with each set of perforations being circumferentially spaced from each other.

The sleeve 290 can be made from any of various suitable materials, including any of various suitable biocompatible polymers, metals or metal alloys. Suitable polymers include, for example, polyurethane, silicone, polyester, nylon, Pebax®, etc., or any of the materials mentioned above that can be used for forming the main body of the balloon. Suitable metals and metal alloys include, for example, stainless steel, Nitinol, or a cobalt chromium alloy. In some examples, the plurality of fingers 292 and the cylindrical portion 294 can be formed from the same material. In some examples, the plurality of fingers 292 and/or the cylindrical portion 294 can be formed from the same material as the main body 280 of the catheter balloon 218. In some examples, the plurality of fingers 292 and/or the cylindrical portion 294 can be integrally formed with the main body 280 of the catheter balloon 218.

FIGS. 4A-4B show a side view of the catheter balloon 218 experiencing an axial tear 288. As shown in FIG. 4A, the axial tear 288 can form in the main body 280 (for example, along the proximal conical portion 282) when the catheter balloon 218 is overinflated. As shown in FIG. 4B, the axial tear 288 can propagate in the proximal direction (for example, in the direction of arrow 289) along the proximal neck portion 281. As the axial tear 288 propagates in the proximal direction, the axial tear 288 can propagate through the gap 293 formed between circumferentially adjacent ones of the plurality of fingers 292. Since the portions of the catheter balloon 218 reinforced by the circumferentially adjacent ones of the plurality of fingers 292 can be radially thicker and consequently more resistant to tearing than unreinforced portions of the catheter balloon 218 (for example, the region corresponding to the gap 293), the axial tear 288 can be more likely to propagate in the axial direction along the gap 293 than in the circumferential direction across one or more of the plurality of fingers 292. Thus, the sleeve 290 can beneficially help control the direction in which the axial tear 288 propagates and can help reduce the likelihood of circumferential tears.

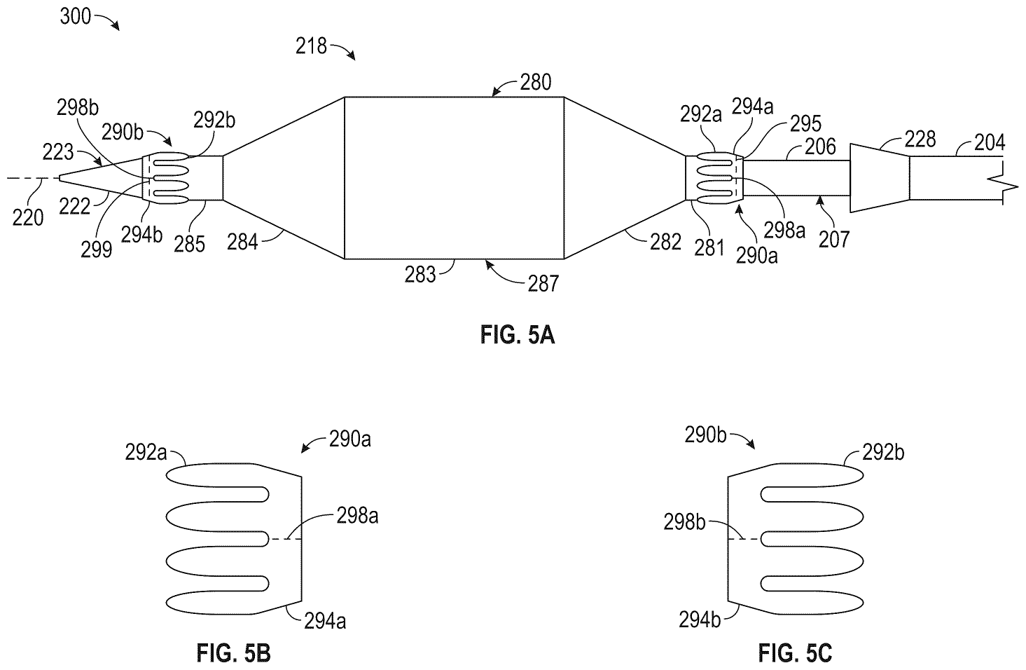

FIG. 5A shows a side view of a distal end portion of a delivery apparatus 300 comprising the catheter balloon 218, according to one example. One exemplary difference between the delivery apparatus 200 of FIG. 3A and the delivery apparatus 300 is that the delivery apparatus 300 can comprise a plurality of sleeves 290. For example, the illustrated delivery apparatus 300 comprises a first sleeve 290a (which may also be referred to herein as a “first catheter balloon sleeve”) disposed at a first end portion of the catheter balloon 218 and a second sleeve 290b (which may also be referred to herein as a “second catheter balloon sleeve”) disposed at a second end portion of the catheter balloon 218. In some examples, the first end portion can be a proximal end portion of the catheter balloon 218 and the second end portion can be a distal end portion of the catheter balloon 218. Thus, the first sleeve 290a can beneficially help control the propagation of tears formed at the proximal end portion of the catheter balloon 218, while the second sleeve 290b can beneficially help control the propagation of tears formed at the distal end portion of the catheter balloon 218.

The first sleeve 290a can comprise a plurality of fingers 292a and a cylindrical portion 294a. In some examples, the plurality of fingers 292a of the first sleeve 290a can share certain similar features with the plurality of fingers 292 and the cylindrical portion 294a can share certain similar features with the cylindrical portion 294. As shown in FIG. 5B, the first sleeve 290a can optionally comprise one or more perforations 298a, which can share certain similar features with the perforations 298.

The plurality of fingers 292a of the first sleeve 290a can partially extend over the proximal end portion of the catheter balloon 218. In the illustrated example, the plurality of fingers 292a partially extend over the proximal neck portion 281. However, the plurality of fingers 292a can additionally or alternatively partially extend over the proximal conical portion 282 and/or the intermediate section 283. The plurality of fingers 292a can be coupled to the proximal end portion of the catheter balloon 218. In some examples, the plurality of fingers 292a can be secured to the catheter balloon 218 with an adhesive, a mechanical fastener, an ultrasonic weld, etc.

In the illustrated example, the cylindrical portion 294a of the first sleeve 290a extends at least partially over the proximal neck portion 281 and the first adjacent surface (for example, the outer surface 207 of the intermediate shaft 206) of the delivery apparatus 300 to cover the proximal terminal end 295 of the proximal neck portion 281. However, the cylindrical portion 294a can additionally or alternatively extend at least partially over the proximal neck portion 281, the proximal conical portion 282, and/or the intermediate portion 283. The cylindrical portion 294a can be coupled to the first adjacent surface. In some examples, the cylindrical portion 294a can be secured to the first adjacent surface with an adhesive, a mechanical fastener, an ultrasonic weld, etc.

In the illustrated example, the second sleeve 290b comprises a first end portion comprising a plurality of fingers 292b and a second end portion comprising a cylindrical portion 294b. In some examples, the plurality of fingers 292b can share certain similar features with the plurality of fingers 292 and the cylindrical portion 294b can share certain similar features with the cylindrical portion 294. In some examples, the second sleeve 290b can share certain similar characteristics with the sleeve 290 and/or the first sleeve 290a. For example, the second sleeve 290b can be identical to the first sleeve 290a.

As shown in FIG. 5C, the second sleeve 290b can optionally comprise one or more perforations 298b, which can share certain similar features with the perforations 298.

The plurality of fingers 292b of the second sleeve 290b can partially extend over the distal end portion of the catheter balloon 218. As shown, the plurality of fingers 292b can partially extend over the distal neck portion 285. However, the plurality of fingers 292b can additionally or alternatively partially extend over the distal conical portion 284 and/or the intermediate section 283. The plurality of fingers 292b can be coupled to the distal end portion of the catheter balloon 218. In some examples, the plurality of fingers 292b can be secured to the catheter balloon 218 with an adhesive, a mechanical fastener, an ultrasonic weld, etc.

The cylindrical portion 294b of the second sleeve 290b can extend at least partially over a second adjacent surface of the delivery apparatus 200. In the illustrated example, the cylindrical portion 294b of the second sleeve 290b extends partially over the distal neck portion 285 and partially over the second adjacent surface (for example, the outer surface 223 of the nose cone 222) such that a distal terminal end 299 of the distal neck portion 285 is located underneath and covered by the cylindrical portion 294b. However, the cylindrical portion 294b can additionally or alternatively extend at least partially over the intermediate portion 283, the distal conical portion 284, and/or the distal neck portion 285. The cylindrical portion 294b can be coupled to the second adjacent surface. In some examples, the cylindrical portion 294b can be secured to the second adjacent surface with an adhesive, a mechanical fastener, an ultrasonic weld, etc.

Some examples of the delivery apparatus 300 can omit the first sleeve 290a. Thus, such examples of the delivery apparatus 300 can comprise the second sleeve 290b disposed on the distal end portion of the catheter balloon 218 without the first sleeve 290a disposed on the proximal end portion of the catheter balloon 218.

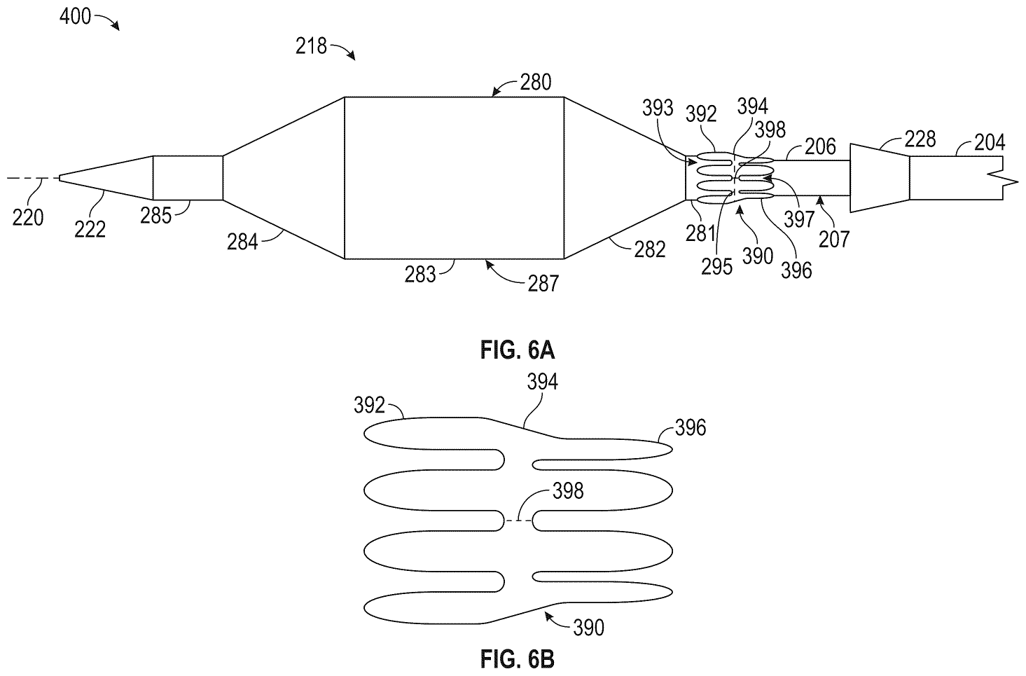

FIG. 6A is a side view of a distal end portion of a delivery apparatus 400 comprising the catheter balloon 218 and a catheter balloon sleeve 390 (which may also be referred to herein as a “sleeve”), according to one example. The sleeve 390 can comprise a first end portion and a second end portion. The first end portion can comprise a first plurality of fingers 392 extending in the axial direction (for example, the distal direction). In some examples, the first plurality of fingers 392 can share certain similar characteristics with the plurality of fingers 292. Circumferentially adjacent ones of the first plurality of fingers can form axially oriented gaps 393 therebetween. In some examples, the gaps 393 can share certain similar characteristics with the gaps 293.

One exemplary difference between the sleeve 290 of FIG. 3A and the sleeve 390 is that the second end portion of the sleeve 390 can comprise an intermediate portion 394 and a second plurality of fingers 396. The intermediate portion 394 can comprise an annular structure, such as in the form of a ring or a cylindrical portion, configured to extend at least partially over an adjacent surface of the delivery apparatus 400. In the illustrated example, the intermediate portion 394 extends at least partially over the proximal neck portion 281 and the first adjacent surface of the delivery apparatus 400 (for example, the outer surface 207 of the intermediate shaft 206) to cover the proximal terminal end 295 of the proximal neck portion 281. In some examples, the intermediate portion 394 can additionally or alternatively be configured to extend at least partially over an end portion of the catheter balloon 218. For example, the intermediate portion 394 can be disposed at least partially over the proximal neck portion 281, the proximal conical portion 282, and/or the intermediate portion 283. In some examples, the intermediate portion 394 can share certain similar characteristics with the cylindrical portion 294.

As shown in FIG. 6B, the intermediate portion 394 can optionally comprise one or more perforations 398. The perforations 398 can be arrayed along an imaginary line extending along a partial or entire axial length of the intermediate portion 394. In some examples, the perforations 398 can share certain similar characteristics with the perforations 298.

The second plurality of fingers 396 can comprise a plurality of circumferentially spaced fingers. As shown, the second plurality of fingers 396 can extend in the axial direction (for example, the proximal direction) from the intermediate portion 394 in a direction opposite the first plurality of fingers 392. Each of the second plurality of fingers 396 can have a radial thickness, a circumferential width, and an axial length. In some examples, at least one of the second plurality of fingers 396 can have a uniform radial thickness and/or a uniform circumferential width along its axial length. In some examples, at least one of the second plurality of fingers 396 can radially and/or circumferentially taper in the direction opposite the first plurality of fingers 392 (for example, in the proximal direction). In the illustrated example, the second plurality of fingers 396 have uniform axial lengths. In some examples, a first set of the second plurality of fingers 396 can have a first axial length and a second set of the second plurality of fingers 396 can have a second, different axial length.

In some examples, each of the first plurality of fingers 392 can have a first radial thickness, the intermediate portion 394 can have a second radial thickness, and each of the second plurality of fingers 396 can have a third radial thickness. In some examples, the first radial thickness can be greater than the second radial thickness. In some examples, the third radial thickness can be greater than the second radial thickness. If the second radial thickness is less than either the first radial thickness or the third radial thickness, the intermediate portion 394 can be weaker than the first and second pluralities of fingers 392 and 396. This configuration can beneficially increase the likelihood that tears will propagate across the intermediate portion 394 in the axial direction instead of across the first or second pluralities of fingers 392 and 396 in the circumferential direction.

Since the second plurality of fingers 396 are circumferentially spaced apart, adjacent ones of the second plurality of fingers 396 can form a second gap 397 therebetween. The gap 397 can extend in the axial direction. In some examples, the gap 397 can share certain similar characteristics with the gap 293 or the gap 393. In some examples, the gap 397 can be circumferentially aligned with the gap 393.

The second plurality of fingers 396 can be configured to extend partially over the adjacent surface of the delivery apparatus 400. In the illustrated example, the second plurality of fingers 396 are arranged in a circumferential direction around a portion of the outer surface 207 of the intermediate shaft 206. In some examples, the second plurality of fingers 396 can additionally extend partially over an end portion of the catheter balloon 218, such as the proximal neck portion 281, the proximal conical portion 282, and/or the intermediate portion 283. The second plurality of fingers 396 can be coupled to the adjacent surface. In some examples, the second plurality of fingers 396 can be secured to the adjacent surface with an adhesive, a mechanical fastener, an ultrasonic weld, etc.

In some examples, each of the first plurality of fingers 392 can be aligned with a corresponding one of the second plurality of fingers 396 in the circumferential direction. When the first plurality of fingers 392 and the second plurality of fingers 396 are circumferentially aligned, an axial tear (such as axial tear 288) can propagate along the gap 393 and across the intermediate portion 394 or the perforation of the intermediate portion 394 in the axial direction. In some examples, this alignment can beneficially help control the axial propagation of the tear.

In some examples, the first plurality of fingers 392 and the second plurality of fingers 396 can have the same size, shape, and orientation such that the sleeve 390 is symmetrical about the intermediate portion 394.

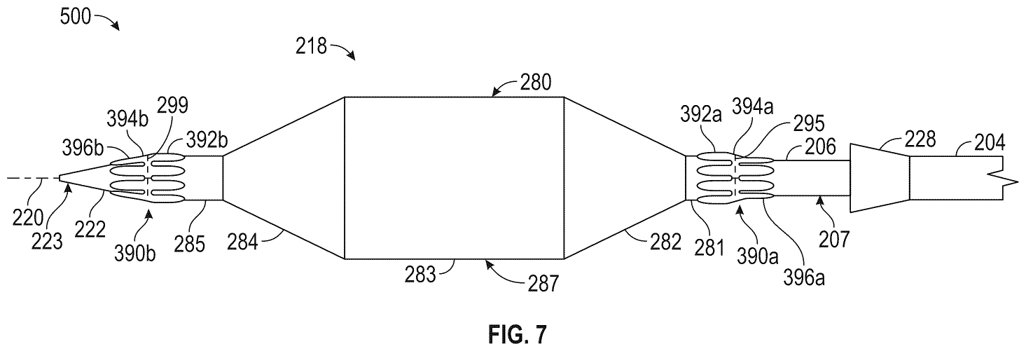

FIG. 7 shows a side view of a distal end portion of a delivery apparatus 500 comprising the catheter balloon 218, according to one example. One exemplary difference between delivery apparatuses 300 and 400 and the delivery apparatus 500 is that the delivery apparatus 500 can comprise a plurality of sleeves 390, wherein each sleeve comprises two pluralities of fingers. For example, the illustrated delivery apparatus 500 can comprise a first sleeve 390a (which may also be referred to herein as a “first catheter balloon sleeve”) disposed at the first end portion of the catheter balloon 218 and a second sleeve 390b (which may also be referred to herein as a “second catheter balloon sleeve”) disposed at the second end portion of the catheter balloon 218. The first end portion can be the proximal end portion of the catheter balloon 218 and the second end portion can be the distal end portion of the catheter balloon 218.

In the illustrated example, the first sleeve 390a comprises a first plurality of fingers 392a, a second plurality of fingers 396a extending in the direction opposite the first plurality of fingers 392a, and an intermediate portion 394a disposed therebetween. In the illustrated example, the intermediate portion 394a extends partially over the proximal neck portion 281 and partially over the outer surface 207 of the intermediate shaft 206 such that a proximal terminal end 295 of the neck portion 281 is located underneath and covered by the intermediate portion 294b. In some examples, the first sleeve 390a can share certain similar characteristics with the sleeve 390 of FIG. 6A.

The second sleeve 390b can comprise a first plurality of fingers 392b, a second plurality of fingers 396b extending in the direction opposite the first plurality of fingers 392b, and an intermediate portion 394b disposed therebetween. In the illustrated example, the intermediate portion 394b of the second sleeve 390b extends partially over the distal neck portion 285 and partially over the second adjacent surface (for example, the outer surface 223 of the nose cone 222) such that a distal terminal end 299 of the distal neck portion 285 is located underneath and covered by the intermediate portion 394b. In some examples, the second sleeve 390b can share certain similar characteristics with the sleeve 390 of FIG. 6A and/or the first sleeve 390a. In some examples, the second sleeve 390b can be identical to the first sleeve 390a.

The second plurality of fingers 396b of the second sleeve 390b can be coupled to the second adjacent surface of the delivery apparatus 500. As shown, the second adjacent surface can be the outer surface 223 of the nose cone 222. However, the second plurality of fingers 396b can additionally or alternatively extend at least partially over the intermediate portion 283, the distal conical portion 284, and/or the distal neck portion 285. The second plurality of fingers 396b can be coupled to the second adjacent surface. In some examples, the second plurality of fingers 396b can be secured to the second adjacent surface with an adhesive, a mechanical fastener, an ultrasonic weld, etc. In some examples, the intermediate portion 394b of the second sleeve 390b can additionally be coupled to the second adjacent surface.

Some examples of the delivery apparatus 500 can omit the first sleeve 390a. Thus, such examples of the delivery apparatus 500 can comprise the second sleeve 390b disposed on the distal end portion of the catheter balloon 218, without the first sleeve 390a disposed on the proximal end portion of the catheter balloon 218.

Some examples of the delivery apparatus 500 can comprise axially arrayed perforations on one of or both the first sleeve 390a and the second sleeve 390b. Such perforations can be similar to the perforations 298 shown in FIG. 6B.

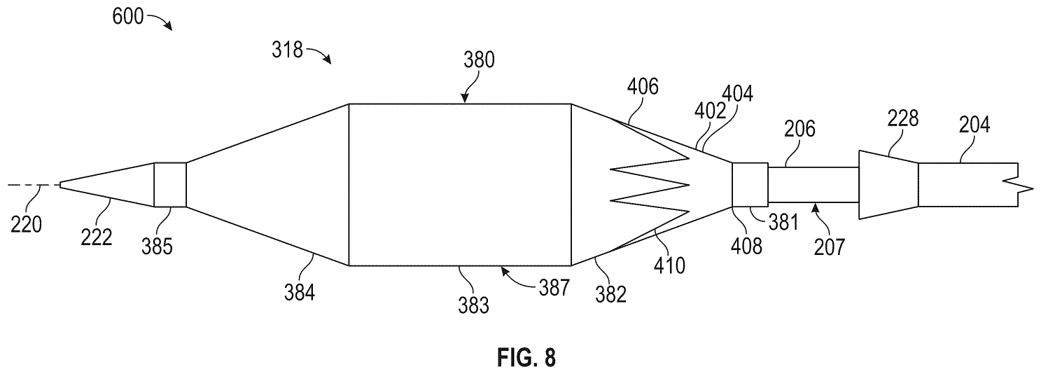

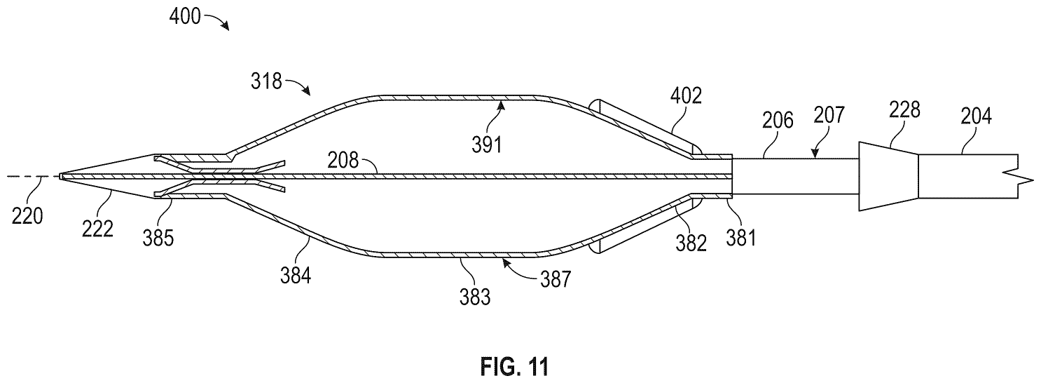

FIG. 8 shows a side view of a distal end portion of a delivery apparatus 600 comprising a catheter balloon 318, according to one example. The catheter balloon 318 can comprise an inflatable main body 380 configured to be inflatable between a deflated state and an inflated state. The main body 380 can comprise a proximal neck portion 381, a proximal conical portion 382 distally disposed relative to the proximal neck portion 381, an intermediate portion 383 distally disposed relative to the proximal conical portion 382, a distal conical portion 384 distally disposed relative to the intermediate portion 383, and a distal neck portion 385 distally disposed relative to the distal conical portion 384. The main body 380 can comprise a circumferential inner surface (FIG. 11) that defines an interior of the catheter balloon 318 and a circumferential outer surface 387 disposed radially outward of the inner surface. Some examples of the main body 380 can share similar characteristics with the main body 280.

The catheter balloon 318 can further comprise a plurality of circumferentially spaced ribs 402. The plurality of ribs 402 can comprise any number of ribs, such as two ribs, three ribs, four ribs five ribs, six ribs, seven ribs, eight ribs, nine ribs, ten ribs, etc. Each of the plurality of circumferentially spaced ribs 402 can extend along at least a portion of the main body 380. As shown, the plurality of ribs 402 extend along a length of a proximal portion of the main body 380, for example, a length of the proximal conical portion 382. In some examples, the plurality of ribs 402 can extend along the entire length of the proximal conical portion 382. In some examples, the plurality of ribs 402 can additionally or alternatively extend along at least a portion of the proximal neck portion 381 and/or the intermediate portion 383.

In the illustrated example, each of the plurality of ribs 402 comprises a first end 404 and a second end 406. The first end 404 of each of the plurality of ribs 402 can be disposed relatively closer to a first end (such as a proximal end) of the main body 380 and the second end 406 of each of the plurality of ribs 402 can be disposed relatively closer to a second end (such as a distal end) of the main body 380.

Each of the plurality of ribs 402 can have an axial length. In the illustrated example, each of the plurality of ribs 402 has the same axial length. In some examples, a first set of the plurality of ribs 402 can have a first axial length and a second set of the plurality of ribs 402 can have a second axial length that is different than the first axial length.

Each of the plurality of ribs 402 can have a circumferential width. In some examples, at least one of the plurality of ribs 402 can have a uniform circumferential width along its axial length. In some examples, at least one of the plurality of ribs 402 can have a first circumferential width at its first end 404 and a second circumferential width at its second end 406. In the illustrated example, the first circumferential width is greater than the second circumferential width. Thus, as shown, the circumferential width of at least one of the plurality of ribs 402 tapers from the first end 404 to the second end 406. In some examples, each of the plurality of ribs 402 can have the same circumferential width. In some examples, a first set of the plurality of ribs 402 can have a first circumferential width and a second set of the plurality of ribs 402 can have a second circumferential width that is different than the first circumferential width.

Each of the plurality of ribs 402 can have a radial thickness. In some examples, at least one of the plurality of ribs 402 can have a uniform radial thickness along its axial length. In some examples, at least one of the plurality of ribs 402 can have a first radial thickness at its first end 404 and a second radial thickness at its second end 406. In some examples, the first radial thickness can be greater than the second radial thickness. In such examples, the radial thickness of the at least one of the plurality of ribs 402 can taper from the first end 404 to the second end 406. In some examples, each of the plurality of ribs 402 can have the same radial thickness. In some examples, a first set of the plurality of ribs 402 can have a first radial thickness and a second set of the plurality of ribs 402 can have a second radial thickness that is different than the first radial thickness.

In some examples, the plurality of ribs 402 can converge at their first ends 404. As used herein, the plurality of ribs 402 “converge” when one end (for example, the first end 404) of one of the plurality of ribs 402 contacts corresponding ends (for example, the first ends 404) of circumferentially adjacent ones of the plurality of ribs 402. As such, the term “converge” does not require that one of the plurality of ribs 402 contacts each of the other ones of the plurality of ribs 402. In the illustrated example, the first ends 404 of the plurality of ribs 402 converge at a conical junction 408 of the proximal neck portion 381 and the proximal conical portion 382. In some examples, the first ends 404 of the plurality of ribs 402 can converge at the proximal neck portion 381. In some examples, the first ends 404 of the plurality of ribs 402 can converge at the proximal conical portion 382. In some examples, the first ends 404 of the plurality of ribs 402 can converge at the intermediate portion 383.

Circumferentially adjacent ones of the plurality of ribs 402 can form a slit 410 therebetween. The slit 410 can extend in the axial direction. The slit 410 can have a circumferential width. In some examples where the circumferentially adjacent ones of the plurality of ribs 402 taper in circumferential width from the first end 404 to the second end 406 of the rib 402, the slit 410 can taper in circumferential width in an opposite axial direction. For example, if the adjacent ones of the plurality of ribs 402 taper in circumferential width in the distal direction, the slit 410 can taper in circumferential width in the proximal direction.

The catheter balloon 318 can be configured direct a tear formed in the main body 380 to propagate along (or in the direction of) the axially extending slit 410. In some examples, portions of the main body 380 that are reinforced by the plurality of ribs 402 can be more tear-resistant than a portion of the main body 380 corresponding to the slit 410. For example, reinforcing portions of the main body 380 with the plurality of ribs 402 can result in the reinforced portions of the main body 380 being radially thicker than unreinforced portions of the main body 380. Additionally or alternatively, the plurality of ribs 402 can be formed of a stronger, more tear-resistant material than the material forming the main body 380. Thus, when a tear forms in the catheter balloon 318, the tear can be more likely to propagate in the proximal direction along the slit 410 and can be less likely to propagate in the circumferential direction across one of the plurality of ribs 402.

The plurality of ribs 402 can be formed of any of various suitable thermoplastics and thermoset polymers. Examples of thermoplastics include polyolefins, polyamides, such as nylon 12, nylon 11, nylon 6/12, nylon 6, and nylon 66, polyesters, polyethers, polyurethanes, polyureas, polyvinyls, polyacrylics, fluoropolymers, copolymers and block copolymers thereof, such as block copolymers of polyether and polyamide, for example, Pebax®; and mixtures thereof. Examples of thermosets include elastomers such as EPDM, epichlorohydrin, nitrile butadiene elastomers, silicones, etc. Thermosets, such as epoxies and isocyanates, can also be used, Biocompatible thermosets may also be used, and these include, for example, biodegradable polycaprolactone, poly(dimethylsiloxane) containing polyurethanes and ureas, and polysiloxanes. In some examples, the plurality of ribs 402 can be formed of the same material as the main body 380 of the catheter balloon 318. In some examples, the ribs can be integrally formed with the main body of the balloon, such as by forming the ribs on the main body during a molding process.

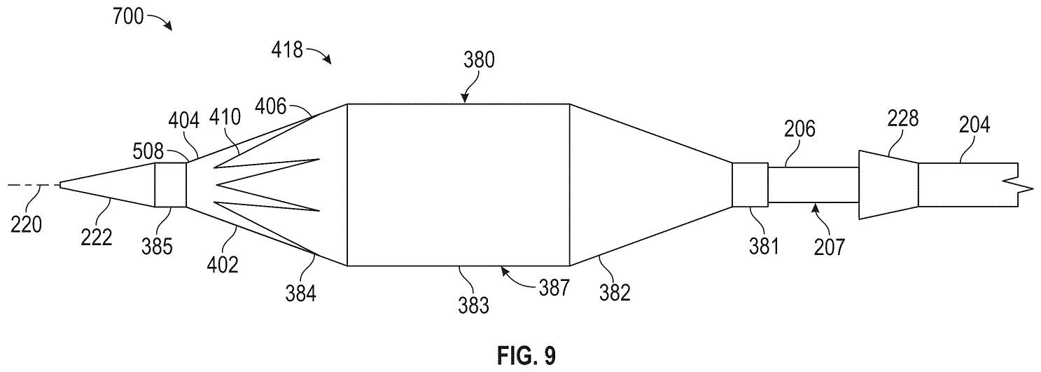

FIG. 9 shows a side view of a distal end portion of a delivery apparatus 700 comprising a catheter balloon 418, according to one example. One exemplary difference between the catheter balloon 318 of the FIG. 8 and the catheter balloon 418 is that the plurality of ribs 402 can extend along a length of a distal portion of the main body 380. As shown, the plurality of ribs 402 extend along a length of the distal conical portion 384. In some examples, the plurality of ribs 402 can extend along the entire length of the distal conical portion 382. In some examples, the plurality of ribs 402 can additionally or alternatively extend along at least a portion of the intermediate portion 383 and/or the distal neck portion 385.

The first end 404 of each of the plurality of ribs 402 can be disposed relatively closer to the distal end of the main body 380 and the second end 406 of each of the plurality of ribs 402 can be disposed relatively closer to the proximal end of the main body 380. As shown, the circumferential width and/or the radial thickness of at least one of the plurality of ribs 402 can taper from the first end 404 to the second end 406. In some examples, at least one of the plurality of ribs 402 can have a uniform circumferential width and/or a uniform radial thickness along its axial length.

In the illustrated example, the first ends 404 of the plurality of ribs 402 converge at a conical junction 508 of the distal conical portion 384 and the distal neck portion 385. In some examples, the first ends 404 of the plurality of ribs 402 can converge at the intermediate portion 383. In some examples, the first ends 404 of the plurality of ribs 402 can converge at the distal conical portion 384. In some examples, the first ends 404 of the plurality of ribs 402 can converge at the distal neck portion 385.

The catheter balloon 418 can be configured direct a tear formed in the main body 380 to propagate along the axially extending slit 410 in the distal direction. Since portions of the main body 380 that are reinforced by the plurality of ribs 402 can be more tear-resistant than a portion of the main body 380 corresponding to the slit 410, any tears formed in the catheter balloon 418 can be more likely to propagate in the distal direction along the slit 410 and can be less likely to propagate in the circumferential direction across one of the plurality of ribs 402.

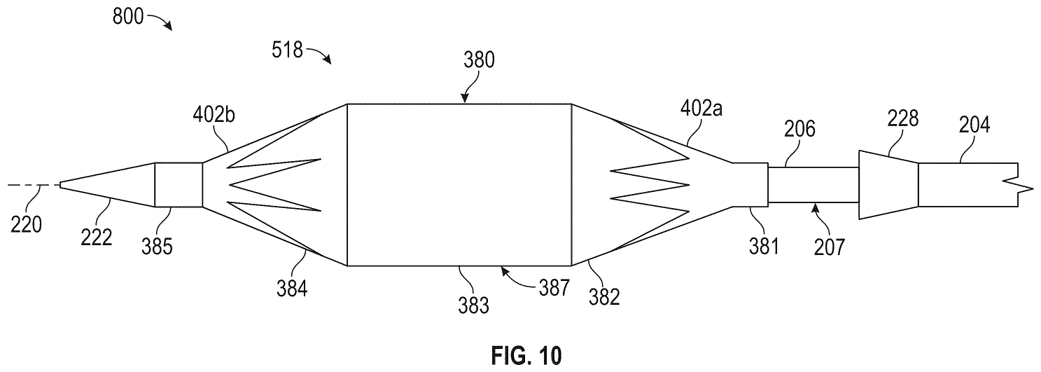

FIG. 10 shows a side view of a distal end portion of a delivery apparatus 800 comprising a catheter balloon 518, according to one example. One exemplary difference between the catheter balloon 518 and catheter balloons 318 and 418 is that the catheter balloon 518 comprises a first plurality of ribs 402a extending along a length of the proximal portion of the main body 380 and a second plurality of ribs 402b extending along a length of the distal portion of the main body 380. In some examples, the first and second pluralities or ribs 402a and 402b can share certain similar characteristics with the plurality of ribs 402.

FIG. 11 shows a cross-section of the distal end portion of the delivery apparatus 600 of FIG. 8, according to a first example. The main body 380 can comprise the circumferential inner surface 391 that defines the interior of the catheter balloon 318 and the circumferential outer surface 387 disposed radially outward of the inner surface 391. As shown, the plurality of ribs 402 extend from the outer surface 387 of the main body 380 in a radially outwards direction and axially along the outer surface 387.

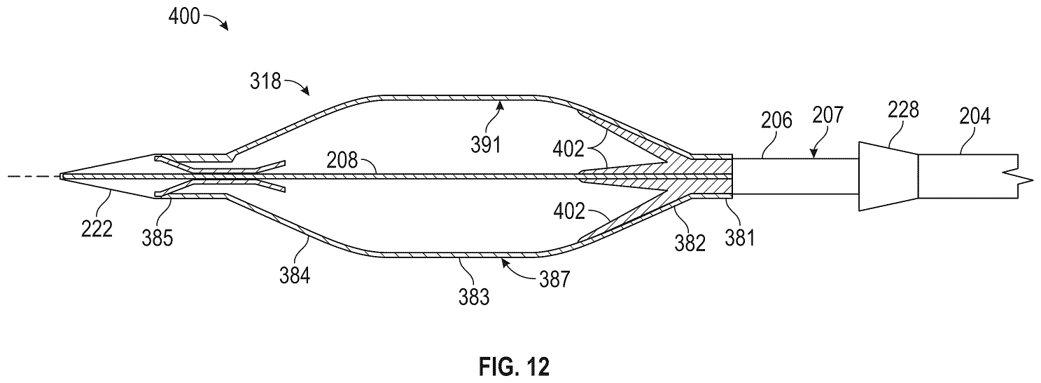

FIG. 12 shows a cross-section of the distal end portion of the delivery apparatus 600 of FIG. 8, according to a second example. As shown, the plurality of ribs 402 extend from the inner surface 391 of the main body 380 in a radially inwards direction and axially along the inner surface.

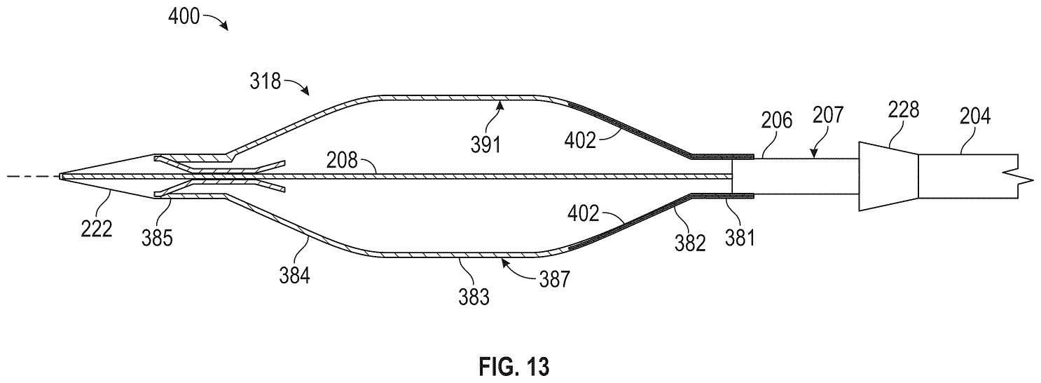

FIG. 13 shows a cross-section of the distal end portion of the delivery apparatus 600 of FIG. 8, according to a third example. As shown, the plurality of ribs 402 are disposed between the inner surface 391 and the outer surface 387 of the main body 380. In some examples, the plurality of ribs 402 can be formed of a more tear-resistant material (for example, a stronger and/or harder material) than the main body 380 to prevent circumferential tears from forming across the plurality of ribs 402.

In some examples, a first set of the plurality of ribs 402 can extend radially outwards from the outer surface 387 and a second set of the plurality of ribs 402 can extend radially inwards from the inner surface 391.

In some examples, a first set of the plurality of ribs 402 can extend radially outwards from the outer surface 387 and a second set of the plurality of ribs 402 can be disposed between the inner surface 391 and the outer surface 387.

In some examples, a first set of the plurality of ribs 402 can extend radially inwards from the inner surface 391 and a second set of the plurality of ribs 402 can be disposed between the inner surface 391 and the outer surface 387.

In some examples, at least one of the plurality of ribs 402 can comprise a radially facing innermost surface and a radially facing outermost surface. The innermost surface can extend radially inwards from the inner surface 391. The outermost surface can extend radially outwards from the outer surface 387.

In some examples, the plurality of ribs 402 formed on the distal end portion of catheter balloons 418 and 518 (FIGS. 9-10) can extend radially outwards from the outer surface 387 in the manner shown for the plurality of ribs 402 in FIG. 11. In some examples, the plurality of ribs 402 formed on the distal end portion of catheter balloons 418 and 518 can extend radially inwards from the inner surface 391 in the manner shown for ribs 402 in FIG. 12. In some examples, the plurality of ribs 402 formed on the distal end portion of catheter balloons 418 and 518 can be disposed between the inner surface 391 and the outer surface 387 in the manner shown for ribs 402 in FIG. 13.

Any one of the first plurality of ribs 402a and the second plurality of ribs 402b shown in FIG. 10 can extend radially outwards from the outer surface 387, extend radially inwards from the inner surface 391, or be disposed between the inner surface 391 and the outer surface 387.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A balloon catheter comprising:
a shaft having a proximal end portion and a distal end portion;
an inflatable balloon mounted on the distal end portion of the shaft, the balloon comprising a main body having first and second end portions; and
a sleeve comprising a first end portion extending partially over the first end portion of the balloon and a second end portion extending partially over an adjacent surface of the balloon catheter, wherein the first end portion of the sleeve comprises a plurality of circumferentially spaced, axially extending fingers.

Example 2. The balloon catheter of any example herein, particularly example 1, wherein the first and second end portions of the balloon are cylindrical, and the balloon comprises an intermediate portion, a first conical portion extending from one end of the intermediate portion to the first end portion of the balloon, and a second conical portion extending from another end of the intermediate portion to the second end portion of the balloon.

Example 3. The balloon catheter of any example herein, particularly any one of examples 1-2, wherein the plurality of fingers comprises a plurality of first fingers, and wherein the second end portion of the sleeve comprises a second plurality of circumferentially spaced, axially extending fingers extending partially over the adjacent surface of the balloon catheter.

Example 4. The balloon catheter of any example herein, particularly example 3, wherein the sleeve further comprises an intermediate portion positioned between and separating the first plurality of fingers and the second plurality of fingers.

Example 5. The balloon catheter of any example herein, particularly any one of examples 1-4, wherein the adjacent surface of the balloon catheter is an outer surface of the distal end portion of the shaft.

Example 6. The balloon catheter of any example herein, particularly any one of examples 1-5, wherein the first end portion of the balloon is a proximal end portion of the balloon.

Example 7. The balloon catheter of any example herein, particularly any one of examples 1-4, wherein the first end portion of the balloon is a distal end portion of the balloon.

Example 8. The balloon catheter of any example herein, particularly any one of examples 1-4, wherein the adjacent surface of the balloon catheter is a surface of a nosecone of the balloon catheter.

Example 9. The balloon catheter of any example herein, particularly any one of examples 1-8, wherein the sleeve is secured to the balloon and the adjacent surface of the balloon catheter with an adhesive.

Example 10. The balloon catheter of any example herein, particularly any one of examples 1-2, wherein:
the sleeve is a first sleeve,
the adjacent surface of the balloon catheter is a first adjacent surface of the balloon catheter, and
the balloon catheter further comprises a second sleeve comprising a first end portion extending partially over the second end portion of the balloon and a second portion extending partially over a second adjacent surface of the balloon catheter, wherein the first end portion of the second sleeve comprises a plurality of circumferentially spaced, axially extending fingers.

Example 11. A sleeve for a catheter balloon comprising:
a cylindrical portion configured to be disposed around an adjacent surface of the balloon catheter and a plurality of circumferentially spaced, axially extending fingers extending from the cylindrical portion.

Example 12. The sleeve of any example herein, particularly example 11, wherein the plurality of fingers are configured to be coupled to an outer surface of the catheter balloon.

Example 13. The sleeve of any example herein, particularly any one of examples 11-12, wherein the plurality of fingers comprises a plurality of first fingers, and wherein the sleeve further comprises a second plurality of circumferentially spaced, axially extending fingers extending from the cylindrical portion in a direction opposite the plurality of first fingers.

Example 14. The sleeve of any example herein, particularly example 13, wherein each of the first plurality of fingers is aligned with a corresponding one of the second plurality of fingers in a circumferential direction.

Example 15. The sleeve of any example herein, particularly any one of examples 11-14, wherein the cylindrical portion has a first radial thickness, each finger has a second radial thickness, and wherein the first radial thickness is less than the second radial thickness.

Example 16. The sleeve of any example herein, particularly any one of examples 11-15, wherein the cylindrical portion comprises an axially extending perforation.

Example 17. A balloon catheter comprising:
a shaft having a proximal end portion and a distal end portion; and
an inflatable balloon mounted on the distal end portion of the shaft, the inflatable balloon comprising:
a main body; and
a plurality of circumferentially spaced ribs extending axially along at least a portion of the main body, wherein each of the plurality of ribs has a first width at a first end thereof and a second width at a second end thereof, wherein the first width is greater than the second width.

Example 18. The balloon catheter of any example herein, particularly example 17, wherein the main body comprises a cylindrical intermediate portion, a proximal conical portion and a distal conical portion, wherein the plurality of ribs extend axially along at least one of the proximal conical portion and the distal conical portion.

Example 19. The balloon catheter of any example herein, particularly example 18, wherein the plurality of ribs extend along the proximal conical portion of the main body.

Example 20. The balloon catheter of any example herein, particularly example 18, wherein the plurality of ribs extend along the distal conical portion of the main body.

Example 21. The balloon catheter of any example herein, particularly any one of examples 17-20, wherein each of the plurality of ribs tapers in width from the first end to the second end of the rib.

Example 22. The balloon catheter of any example herein, particularly any one of examples 17-21, wherein the plurality of ribs converge at the first ends of the ribs.

Example 23. The balloon catheter of any example herein, particularly any one of examples 17-22, wherein the first and second widths are measured in a circumferential direction of the balloon.

Example 24. A balloon catheter comprising:
a shaft having a proximal end portion and a distal end portion; and
an inflatable balloon mounted on the distal end portion of the shaft, the inflatable balloon comprising:
   a main body having first and second end portions; and
   a plurality of circumferentially spaced ribs extending axially along the first end portion of the main body, wherein each rib has a first end disposed toward the first end portion of the main body and a second end disposed toward the second end portion of the main body, wherein the ribs converge at their first ends.

Example 25. The balloon catheter of any example herein, particularly example 24, wherein the main body further comprises an inner surface and an outer surface disposed radially outwards of the inner surface.

Example 26. The balloon catheter of any example herein, particularly example 25, wherein the plurality of ribs extend from the inner surface of the main body in a radially inwards direction.

Example 27. The balloon catheter of any example herein, particularly example 25, wherein the plurality of ribs extend from the outer surface of the main body in a radially outwards direction.

Example 28. The balloon catheter of any example herein, particularly example 25, wherein the plurality of ribs are disposed between the inner surface and the outer surface of the main body.

Example 29. The balloon catheter of any example herein, particularly any one of examples 24-28, wherein adjacent ones of the plurality of ribs from an axially extending slit therebetween, and wherein the adjacent ones of the plurality of ribs are configured to direct a tear formed in the main body of the inflatable balloon to propagate along the axially extending slit.

Example 30. A catheter balloon of any example herein, particularly any one of examples 1-29, wherein the catheter balloon is sterilized.

The features described herein with regard to any example can be combined with other features described in any one or more of the other examples, unless otherwise stated. For example, any one or more of the features of one delivery apparatus can be combined with any one or more features of another delivery apparatus. As another example, any one or more features of one catheter balloon can be combined with any one or more features of another catheter balloon.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A balloon catheter comprising:
a shaft having a proximal end portion and a distal end portion;
an inflatable balloon mounted on the distal end portion of the shaft, the balloon comprising a main body having first and second end portions; and
a sleeve comprising a first end portion extending partially over the first end portion of the balloon and a second end portion extending partially over an adjacent surface of the balloon catheter, wherein the first end portion of the sleeve comprises a plurality of circumferentially spaced, axially extending fingers.

2. The balloon catheter of claim 1, wherein the first and second end portions of the balloon are cylindrical, and the balloon comprises an intermediate portion, a first conical portion extending from one end of the intermediate portion to the first end portion of the balloon, and a second conical portion extending from another end of the intermediate portion to the second end portion of the balloon.

3. The balloon catheter of claim 1, wherein the plurality of fingers comprises a plurality of first fingers, and wherein the second end portion of the sleeve comprises a second plurality of circumferentially spaced, axially extending fingers extending partially over the adjacent surface of the balloon catheter.

4. The balloon catheter of claim 3, wherein the sleeve further comprises an intermediate portion positioned between and separating the first plurality of fingers and the second plurality of fingers.

5. The balloon catheter of claim 1, wherein the adjacent surface of the balloon catheter is an outer surface of the distal end portion of the shaft.

6. The balloon catheter of claim 1, wherein the first end portion of the balloon is a proximal end portion of the balloon.

7. The balloon catheter of claim 1, wherein the first end portion of the balloon is a distal end portion of the balloon.

8. The balloon catheter of claim 1, wherein the adjacent surface of the balloon catheter is a surface of a nosecone of the balloon catheter.

9. The balloon catheter of claim 1, wherein the sleeve is secured to the balloon and the adjacent surface of the balloon catheter with an adhesive.

10. The balloon catheter of claim 1, wherein:
the sleeve is a first sleeve,
the adjacent surface of the balloon catheter is a first adjacent surface of the balloon catheter, and
the balloon catheter further comprises a second sleeve comprising a first end portion extending partially over the second end portion of the balloon and a second portion extending partially over a second adjacent surface of the balloon catheter, wherein the first end portion of the second sleeve comprises a plurality of circumferentially spaced, axially extending fingers.

11. A balloon catheter comprising:
a shaft having a proximal end portion and a distal end portion; and
an inflatable balloon mounted on the distal end portion of the shaft, the inflatable balloon comprising:
a main body; and
a plurality of circumferentially spaced ribs extending axially along at least a portion of the main body, wherein each of the plurality of ribs has a first width at a first end thereof and a second width at a second end thereof, wherein the first width is greater than the second width.

12. The balloon catheter of claim 11, wherein the main body comprises a cylindrical intermediate portion, a proximal conical portion and a distal conical portion, wherein the plurality of ribs extend axially along at least one of the proximal conical portion and the distal conical portion.

13. The balloon catheter of claim 12, wherein the plurality of ribs extend along the proximal conical portion of the main body.

14. The balloon catheter of claim 12, wherein the plurality of ribs extend along the distal conical portion of the main body.

15. The balloon catheter of claim 11, wherein each of the plurality of ribs tapers in width from the first end to the second end of the rib.

16. The balloon catheter of claim 11, wherein the plurality of ribs converge at the first ends of the ribs.

17. The balloon catheter of claim 11, wherein the first and second widths are measured in a circumferential direction of the balloon.

18. A balloon catheter comprising:
a shaft having a proximal end portion and a distal end portion; and
an inflatable balloon mounted on the distal end portion of the shaft, the inflatable balloon comprising:
a main body having first and second end portions; and
a plurality of circumferentially spaced ribs extending axially along the first end portion of the main body, wherein each rib has a first end disposed toward the first end portion of the main body and the second end disposed toward a second end portion of the main body, wherein the ribs converge at their first ends.

19. The balloon catheter of claim 18, wherein the main body further comprises an inner surface and an outer surface disposed radially outwards of the inner surface.

20. The balloon catheter of claim 19, wherein the plurality of ribs extend from the inner surface of the main body in a radially inwards direction.
